(19) United States
(12) Patent Application Publication
(10) Pub. No.: US 2017/0157466 A1
(43) Pub. Date: Jun. 8, 2017

(54) DISPLAYING ACTIVITY DATA

(71) Applicant: POLAR ELECTRO OY, Kempele (FI)

(72) Inventors: Matti KORPELA, Oulu (FI); Olli KOMULAINEN, Oulu (FI); Jukka JAATINEN, Kempele (FI); Taina KOISTINEN, Ylikiiminki (FI)

(21) Appl. No.: 15/039,302

(22) PCT Filed: Nov. 27, 2013
(86) PCT No.: PCT/FI2013/051110

§ 371 Date: May 25, 2016

(51) Int. Cl. A63B 24/00; A61B 5/0205; G06F 19/00; A61B 5/00; G02F 1/163; A63B 71/06; A61B 5/11

(52) U.S. Cl. CPC A63B 24/0062; A63B 71/0686; A61B 5/02055; A61B 5/1118; A61B 5/7445; G02F 1/163; G06F 19/3481; A63B 2230/04; A63B 2220/12; A63B 2220/30; A63B 2220/62; A63B 2220/22; A63B 2220/50; A63B 2220/836; A63B 2230/75; A63B 2220/20; A63B 2220/73; A63B 2220/72; A63B 2220/74; A63B 2024/0065; A63B 2220/803; A63B 2071/0663; A61B 5/02438

(57) ABSTRACT

A portable physical activity monitor device includes a power source for providing power to the physical activity monitor device, at least one processor and at least one memory including a computer program code. The at least one memory and the computer program code are configured, with the at least one processor, to cause the physical activity monitor device to perform operations including acquiring an indication of a physical activity metric of a person carrying the physical activity monitor device. The physical activity monitor device includes at least one electrochromic part including bi-stable electrochromic material, and the physical activity monitor device is caused to control an optical characteristic.

### CROSS-REFERENCE TO RELATED APPLICATION

This application is a National Stage application of International Application No. PCT/FI2013/051110, filed Nov. 27, 2013, which is incorporated by reference herein in its entirety.

### BACKGROUND

Field

The invention relates generally to displaying activity data.

Description of the Related Art

It is important to display activity data in such a manner that the user is quickly able to see the data. Typically, in the field of physical activity monitors, the display of exercise or activity data is by means of characters displayed on a display, such as on a liquid crystal display (LCD) of a training computer which the user may wear in his/her wrist. However, showing the activity/exercise data with relatively small characters on the small display may not be the most user-friendly manner. Moreover, the display may be the most power consuming part of the training computer.

### SUMMARY

According to an aspect of the invention, there is provided a portable physical activity monitor device, comprising: a power source for providing power to the physical activity monitor device; at least one processor and at least one memory including a computer program code, wherein the at least one memory and the computer program code are configured, with the at least one processor, to cause the physical activity monitor device at least to: acquire an indication of a physical activity metric of a person carrying the physical activity monitor device, wherein the physical activity monitor device further comprises at least one electrochromic part comprising bi-stable electrochromic material, and the physical activity monitor device is further caused to: control an optical characteristic of the electrochromic part on the basis of the acquired activity metric.

According to an aspect of the invention, there is provided a computer program product embodied on a non-transient distribution medium readable by a computer and comprising program instructions which, when loaded into a portable physical activity monitor device, cause the physical activity monitor device to execute at least the following: acquire an indication of a physical activity metric of a person carrying the physical activity monitor device, wherein the physical activity monitor device comprises at least one electrochromic part comprising bi-stable electrochromic material; and control an optical characteristic of the electrochromic part on the basis of the acquired activity metric.

According to an aspect of the invention, there is provided a non-transient computer-readable distribution medium carrying the above-mentioned computer program product.

According to an aspect of the invention, there is provided an apparatus comprising means for performing any of the embodiments as described in the appended claims.

Embodiments of the invention are defined in the dependent claims.

### BRIEF DESCRIPTION OF THE DRAWINGS

In the following, the invention will be described in greater detail with reference to the embodiments and the accompanying drawings, in which

FIG. 1 presents a physical activity monitor device (PAMD), according to an embodiment;

FIGS. 2A and 2B show how an electrochromic (EC) material may be controlled, according to an embodiment;

FIG. 3 shows an example of how to control EC parts of the PAMD on the basis of a physical activity metric, according to an embodiment;

FIG. 4 illustrates how the activity zones may be redefined, according to an embodiment;

FIGS. 5A to 5C show an example in which the physical activity metric depicts an accumulated activity level, according to an embodiment;

FIGS. 6A to 6B illustrate an example on how a target activity level may affect the control of the optical states of the EC parts, according to an embodiment;

FIG. 7 shows resetting the accumulated activity levels, according to an embodiment;

FIG. 8 illustrates a layered structure for arranging the EC parts, according to an embodiment;

FIGS. 9A to 9D show some examples for implementing the EC parts to the PAMD, according to some embodiments; and

FIGS. 10A to 10D show an example for implementing the EC part to the PAMD 100, according to an embodiment.

### DETAILED DESCRIPTION

The following embodiments are exemplary. Although the specification may refer to “an”, “one”, or “some” embodiment(s) in several locations of the text, this does not necessarily mean that each reference is made to the same embodiment(s), or that a particular feature only applies to a single embodiment. Single features of different embodiments may also be combined to provide other embodiments.

Typically physical activity data may be displayed on the display of a wrist-worn training computer. However, the user may find the characters small and hard to quickly read the activity data. Further, such active display may be the most power consuming component of the portable training computer. Nevertheless, a proper contrast in the display may be important for clear illustration of the activity data. Therefore it may be beneficial to provide a physical activity monitor device which is able to illustrate the activity data clearly with low battery consumption.

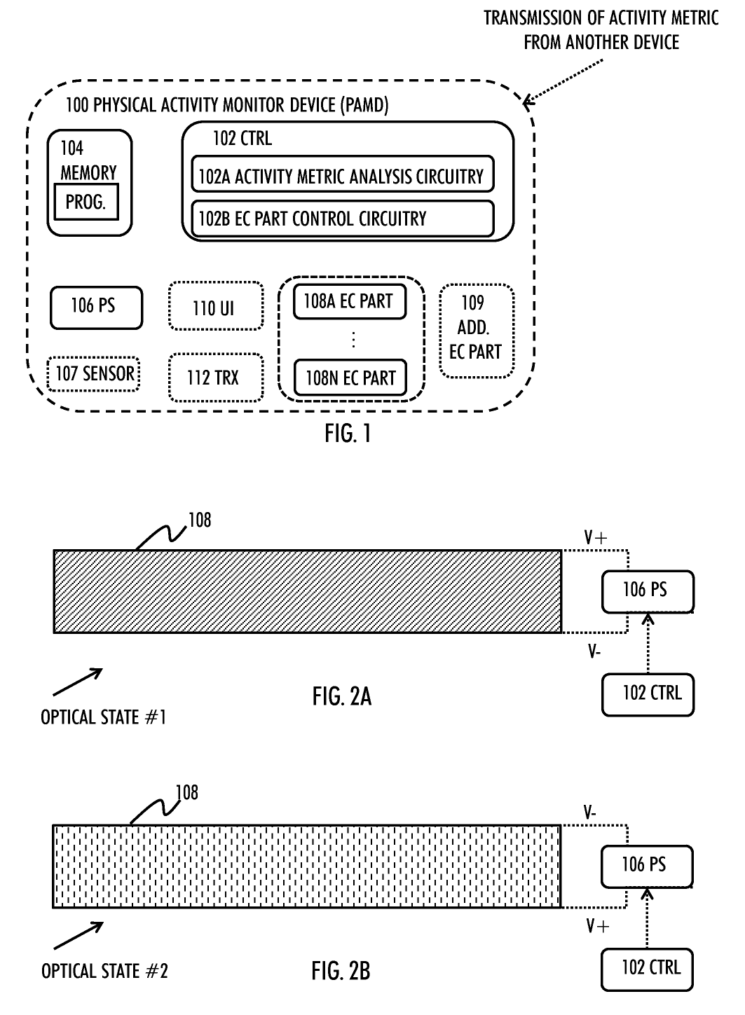

Accordingly, there is provided a portable physical activity monitor device (PAMD) 100, as shown in FIG. 1, which comprises a power source (PS) 106 for proving power to the PAMD 100. The power source may be a replaceable battery, a rechargeable battery, for example. As said, the PAMD 100 may be portable so that the PAMD 100 is carried by the user/person/exerciser. Such portable use case may pose limitations to the PAMD 100. For example, the size of any portable device is limited and so may be the battery power and the processing capabilities.

The PAMD 100 further comprises at least one processor/controller 102 and at least one memory 104 including a computer program code (PROG). The at least one memory 104 and the computer program code are configured, with the at least one processor 102, may be arranged to cause the PAMD 100 to perform various tasks. In an embodiment, the PAMD 100 is caused to acquire an indication of an activity metric of a person carrying the PAMD 100. The activity metric may comprise one or more values representing a physical or physiological activity of the person carrying the PAMD 100. For example, samples of a heart rate or calories consumed may be used as the activity metric. The activity metric may also be called a physiological activity sample, value, metric or measure.

In an embodiment, the PAMD 100 may be or be comprised in an exercise sensor, such as a heart activity sensor, a sensor (e.g. a foot pod or a global positioning system (GPS) receiver) acquiring location or speed/pace specific information, or a sensor acquiring cadence data, for example. In such embodiment where the PAMD 100 is a sensor, the PAMD 100 may itself measure, monitor or determine the activity metric, such as the distance elapsed or heart rate samples, to mention only a few possible options.

In another embodiment, the PAMD 100 may be or be comprised in a training computer, such as a wrist-worn training computer. The training computer may obtain activity metric/data from any of physiological activity sensors carried by the person. Thus, in an embodiment, the PAMD 100 may acquire the activity metric by receiving the metric from at least one of the external sensors, as shown in FIG. 1 with the dotted arrow. In another embodiment, the training computer as the PAMD 100 may acquire the activity metric by determining the activity data/metric itself. This may be the case, for example, when the training computer itself comprises at least one sensor 107, such as an accelerometer, for measuring the at least one activity metric of the person.

Let us now take a closer look at the PAMD 100. The PAMD 100 further comprises at least one electrochromic (EC) part 108A, . . . , 108N (commonly referred with a reference numeral 108). The EC part 108 may comprise bi-stable electrochromic material. As implied by the word “bi-stable”, the EC part 108 may rest in either of the two inherent states without consuming any battery power. The EC part 108 may be disposed/arranged in the PAMD 100 such that the EC part 108 is visible to outside, e.g., to the person carrying the PAMD 100.

The EC parts 108 are capable of electrochromism. Electrochromism is a phenomenon in which the selected EC material reversibly changes the colour, or more generally, in which the selected EC material reversibly changes an optical characteristic. The change in the optical characteristic may be caused by applying a burst of charge to the EC material, as shown in FIGS. 2A and 2B. The change of the optical state may thus be controlled with electricity. The burst of energy may be output from the power source 106 under the control of the controller 102. In FIG. 2A, the optical state of the EC part 108, which may be defined by the optical characteristics of the EC material, is different than in Figure of 2B. This is represented with a difference in the fill patterns of FIGS. 2A and 2B.

The optical characteristic which may be changed may include transparency, colour, shade, reflection related properties, or diffraction related properties of the EC material, for example. The change in the in the optical characteristic may be persistent and energy (e.g. voltage/charge) may be needed only to effect the change in the optical characteristic. This is shown in FIG. 2A in which a certain voltage is provided to the EC part 108 in order to cause the EC material to adopt the desired optical characteristic.

The change of the optical characteristic may be reversible. This denotes that a change from transparency to green, for example, may be reversed (i.e. the EC material may be changed back from green to being transparent). This reversible change may be effected by applying a voltage/charge with a changed polarity to the EC material, as shown in FIG. 2B in which the polarity of the applied voltage is different than in FIG. 2A.

Various types of materials and structures may be used to construct such EC parts capable of electrochromism. These materials comprise, e.g., polyaniline, viologens, polyoxotungstates and tungsten oxide. E.g., if an electrode is immersed in hydrochloric acid which contains a small concentration of aniline, then a film of polyaniline may be grown on the electrode.

In an embodiment, each EC material may have its own specific optical characteristic-pair. One EC material may be controlled to change the optical state between transparency and red, whereas another EC material may be controlled to change the optical state between green and yellow, for example. As an example, the polyaniline may be either pale yellow or dark green/black. Some EC materials change from being a transparent to a single specific colour, such as to green, red, black, orange, or yellow, to mention only a few possible options.

In another embodiment, the same EC material may be caused to change its optical characteristic to many different colours or shades (i.e. the intensity of a colour), depending on what is the level of applied charge to the EC material. Also a gradual change from a transparent state to a certain colour, such as to black, may be possible, wherein the change from comprises multiple different colours/shades in between.

In general, a change to any desired colour is possible with an appropriate EC material by feeding an appropriate amount of voltage/charge to the EC material. As said, the EC material may not need any power for keeping the optical state but only for changing the optical state. Thus, the EC part does not consume much power of the PAMD 100, which may be beneficial for small-size portable devices.

In an embodiment, the at least one memory 104 and the computer program code are configured, with the at least one processor 102, to cause the PAMD 100 further to control the optical characteristic of the at least one EC part 108 on the basis of the acquired activity metric. For example, the colour or transparency may be changed according to the activity metric.

In an embodiment, the control of the optical characteristic comprises a reversible change between a first and a second optical state, wherein, in the first state, the EC material is transparent and, in the second state, the EC material is not transparent.

In another embodiment, the control of the optical characteristic comprises a reversible change between a first and a second optical state, wherein, in the first state, the EC material adopts a first colour and, in the second state, the EC material adopts a different second colour.

As one example, the EC part 108 may be transparent (that is, invisible) when the heart rate (as indicated by the acquired activity metric) is lower than a predetermined threshold. If it is detected that the heart rate increases over the threshold, the EC part 108 may be fed with an appropriate amount of voltage in order to make the EC part 108 to adopt a red colour, for example. The coloured EC part 108 may be visible to the person. As the person detects the colour on the EC part 108, the person may, for example, detect that his/her heart rate is too high. The person may have himself/herself entered the threshold value to the PAMD 100 via a user interface (UI) 110 of the PAMD 100, for example. Such UI 110 may comprise, for example, at least one keypad or button, a microphone, a touch display, a touch display, a speaker, etc.

The proposed PAMD 100 may provide a benefit according to which the person may easily detect a change in the activity metric. As will be explained later, the EC part 108 may occupy a portion of the surface of the PAMD 100. The portion may be a relatively large portion of the total surface of the PAMD 100. The change of the optical state of the EC part 108 may thus be visible in a large portion of the surface of the PAMD 100. Detecting a change of colour in the surface part of the PAMD 100 may be significantly easier than detecting a change of numerical character on a display. Moreover, maintaining the display of the current value of the activity metric with the EC part may not consume any power. Power may be needed only for changing the optical state of the EC part 108 but not for maintaining the optical state. This may be important for small sized devices, such as exercise sensors or training computers.

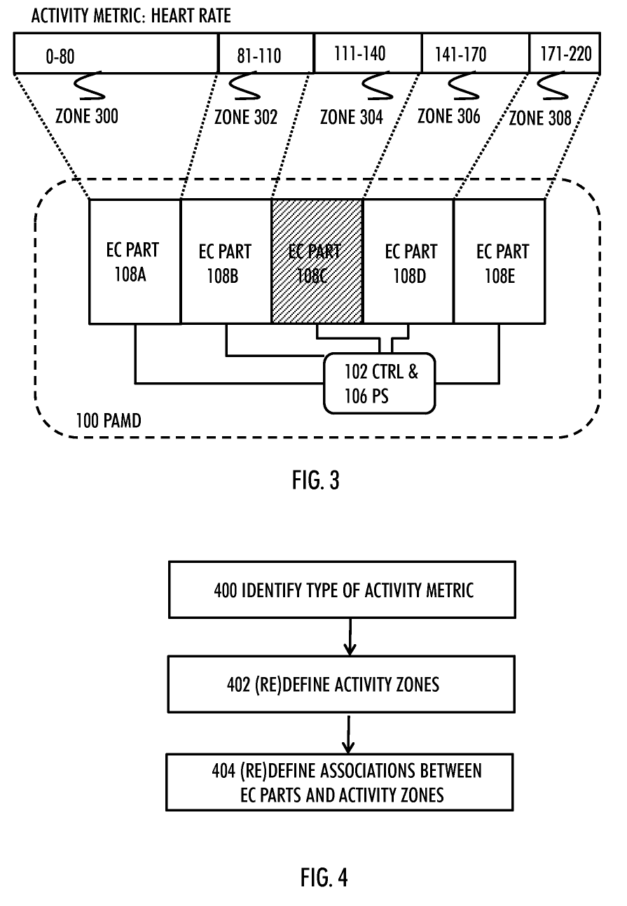

In an embodiment as shown in FIG. 3, the PAMD 100 comprises a plurality of EC parts 108A-108E, each comprising bi-stable electrochromic material. In an embodiment, each of the EC parts 108A-108E is able to adopt a certain optical state which is different from the optical state adoptable by another EC part of the PAMD 100. In another embodiment, at least some of the EC parts 108A-108E are able to adopt the same optical state, such as at least some EC parts may turn into green upon applying certain voltage to the corresponding EC parts.

The PAMD 100 may be caused to define a plurality of activity zones 300 to 308, wherein each activity zone comprises a different range of activity metric values. The different ranges may be non-overlapping. The ranges within each activity zone need not be even. That is, the range of one activity zone may be narrower or wider than the range in another activity zone. As an example, when the activity metric indicates heart rate samples/values, the activity zones may be defined as given in FIG. 3. The first zone 300 comprises heart rates of 0 to 80 pulses per minute (PPM), the second zone 302 comprises heart rates of 81 to 110 PPM, the third zone 304 comprises heart rates of 111 to 140 PPM, the fourth zone 306 comprises heart rates of 141 to 170 PPM, and the fifth zone 308 comprises heart rates of 171 to 220 PPM, for example. There may be more or less than five activity zones defined, five zones are used here simply for illustrative purposes.

In an embodiment, there may be at least as many EC parts 108 as there are activity zones 300-308. The PAMD 100 may then associate each activity zone 300-308 with a different EC part 108A-108E. In the given example of FIG. 3, the physical activity zone 300 is associated with the EC part 108A, the physical activity zone 302 is associated with the EC part 108B, etc.

Then the PAMD 100 may then detect to which of the plurality of activity zones 300-308 the acquired activity metric belongs to. For example, if the currently received heart rate value is 123 PPM, it may be detected that this value belongs to the activity zone 304. The PAMD 100 may further identify which EC part is associated with the activity zone 304 comprising the acquired activity metric. In this case, the identification may reveal that the EC part 108C is associated with the activity zone 304.

As a result, the PAMD 100 may control the optical characteristic of each of the plurality of EC parts 108A-108E on the basis of the identification, that is on the basis of which of the plurality of EC parts 108A-108E is associated with the activity zone 304 to which the acquired activity metric belongs to. As one example, the EC part 108C may be controlled to show a certain colour, while the other EC parts 108A, 108B, 108D and 108E may be maintained transparent/in another colour. That is, the EC part 108C may be highlighted with respect to the other EC parts.

In an embodiment, the PAMD 100 may be caused to identify the type of the activity metric, as shown in step 400 of FIG. 4. This identification may be based on user instructions given via the UI 110. Additionally or instead, the identification may be based at least partially on the activity metric. That is, the PAMD 100 may detect the type of the activity metric. Such detection may be based on identifier acquired from the device transmitting the activity metric, in case the PAMD 100 does not itself determine the activity metric values. The device identifier, such as a MAC identifier identifying a sensor, may imply the type of the sensor, such as heart rate sensor. Alternatively, the acquired activity metric data includes an identifier of the type of the activity metric.

One type of the activity metric may represent instantaneous activity samples, such as heart rate samples, current altitude, current speed/pace, to mention only a few possible examples. Another type of activity metric may represent accumulated values, such as consumed calories, number of steps taken, or distance elapsed, to mention only a few possible examples. Moreover, the PAMD 100 may identify the exact type of the activity metric within the group of accumulated activity metric values or within the group of instantaneous activity metric samples/values.

Accordingly, the PAMD 100 may in step 402 (re)define the plurality of activity zones on the basis of the identified type of activity metric. For example, in case the activity metric depicts the amount of consumed calories, the zones may be different than in case the activity metric would depict the amount of steps taken or than in case the activity metric would depict instantaneous heart rate samples, for example. Moreover, the PAMD 100 may (re)define the associations between the EC parts and the activity zones in step 404.

Each type of activity metric may have predefined activity zones 300-308 and associations between the activity zones 300-308 and the EC parts 108A-108E stored in the memory of the PAMD 100. The PAMD 100 may determine which ones of the stored activity zones 300-308 and associations to take into use on the basis of the identified type of the activity metric. It may be noted that the user may also determine which activity metric is to be displayed with the EC parts 108A-108E.

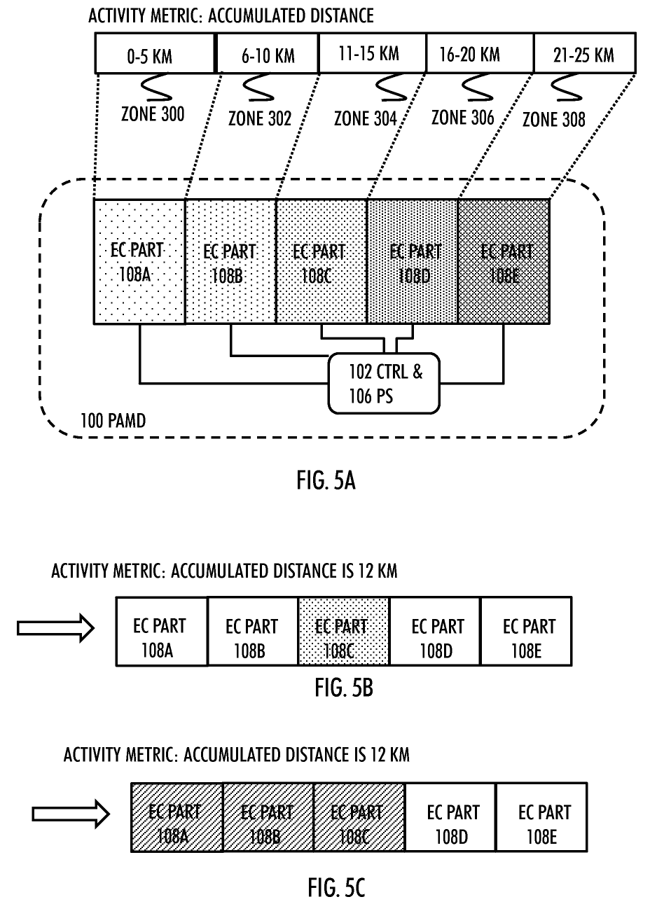

The PAMD 100 may be caused to redefine the ranges of the stored activity zones 300-308 on the basis of user instructions or empirical usage history. This may be beneficial as then the user may modify the activity zones according to his/her own needs. For example, let us assume that original (preconfigured) activity zones 300 to 308 for the accumulated distance comprise a range of kilometres from 0 to 25 in intervals of 5 kilometres (as shown in FIG. 5A). However, the user typically runs only 5 to 10 kilometres. In such case, the user may decide to reconfigure the activity zones 300 to 308 for the accumulated distance to comprise a range of kilometres from 0 to 10 in intervals of 2 kilometres, for example.

In one example, the PAMD 100 may monitor the lengths of the jogs performed by the person over an observation period. The PAMD 100 may learn that the person typically runs between 8 to 12 kilometres. In such case, the PAMD 100 may automatically reconfigure the activity zones 300 to 308 for the accumulated distance to comprise a range of kilometres from 0 to 15, in intervals of 3 kilometres, for example.

In one example, the PAMD 100 may redefine the ranges of the activity zones 300-308 on the basis of physiological tests performed by the person, such as on the basis of OwnZone®-test by Polar Electro®. For example, when the EC parts 108A-108E depict instantaneous heart rate samples, the ranges of the activity zones 300-308 may be defined by the OwnZone®-test.

In an embodiment, as shown in FIG. 5A, the acquired activity metric represents an accumulated activity level (e.g. acquired activity metric carries accumulated activity values). In the example of FIG. 5, the accumulated activity metric is an accumulated distance. Similarly, the accumulated activity metric could indicate another type of activity, such as elapsed time of the training, for example. In this case, each of the plurality of activity zones 300-308 may comprise a different range of accumulated activity levels. The different ranges may be non-overlapping.

As shown in FIG. 5A, different EC parts 108A-108E may be configurable to adopt a different optical state, such as a different colour. In another embodiment, different EC parts 108A-108E may be configurable to adopt a same colour, for example. In one embodiment, the different EC parts 108A-108E may be configurable to change from transparency to having a visible colour (same or different colour among the different EC parts 108A-108E.

The PAMD 100 may control the optical characteristic of each of the plurality of EC parts 108A-108E on the basis of what the accumulated activity level is. Non-limiting examples to the control of the optical characteristic are shown in FIGS. 5B and 5C. In these examples it is assumed that the accumulated distance implied by the activity metric is 12 km. As shown in FIG. 5A, such accumulated value is within the range of the activity zone 304, which is associated with the EC part 108C.

In FIG. 5B, the PAMD 100 may control the optical characteristics of the EC parts 108A-108E such that the EC part 108C, which corresponds to the activity zone 304 comprising the accumulated activity level (i.e. 12 km), adopts a different optical state than the rest of the EC parts 108A, 108B, 108D, and 108E. That is, one of the EC parts 108C is highlighted with respect to the other EC parts. The optical states of the EC parts 108A-108E may be changed one-by-one as the accumulated activity level increases, wherein the previously highlighted EC part (such as EC part 108B) is changed back to another optical state upon highlighting the next EC part (such as EC part 108C). As an example, the EC part 108C may be caused to display a certain visible colour (e.g. green), whereas the rest of the EC parts 108A, 108B, 108D, and 108E may be transparent or display a different colour (e.g. red).

In the embodiment of FIG. 5C, the PAMD 100 may control the optical characteristics of the EC parts 108A-108E such that the at least one EC part, which corresponds to the lower or higher accumulated activity levels than the current accumulated activity level (i.e. 12 km), adopts a different optical state than the rest of the EC parts. For example, the EC parts 108A-108C which are associated with accumulated activity levels which are equal to or lower than 12 km are caused to display a certain colour (e.g. green), whereas the rest of the EC parts 108D and 108E may be transparent or depict a different colour (e.g. red). That is, the optical states of the EC parts 108A-108E may be changed one-by-one as the accumulated activity level increases, wherein the previously highlighted EC part (such as EC part 108B) maintains the current optical state upon changing the optical state of the next EC part (such as EC part 108C). In an example, the EC parts 108A-108C may have the same optical state, such as a colour or transparency. In another example, the EC parts 108A-108C may have different colours or shades. In an example, the other EC parts 108D-108E may be transparent. In another embodiment, the EC parts 108D and 108E are highlighted, whereas the EC parts 108A-108C are not.

In an embodiment, the PAMD 100 may acquire a command to accumulate activity metric values. Such command may be given by the user of the PAMD 100 via the UI 110. Alternatively, the PAMD 100 may detect on the basis of the type of the activity metric that these acquired activity metric values indicate accumulated values or are more appropriately displayed when accumulated. For example, altitude related activity metric may be depicted as instantaneous samples depicting the current altitude in which case the EC parts 108A-108E may depict different altitude ranges above the sea level. However, in another embodiment, the altitude related activity metric may be depicted as accumulated values indicating the amount of meters travelled in a vertical direction. In such case the EC parts 108A-108E may represent different ranges for the accumulated meters travelled in the vertical direction.

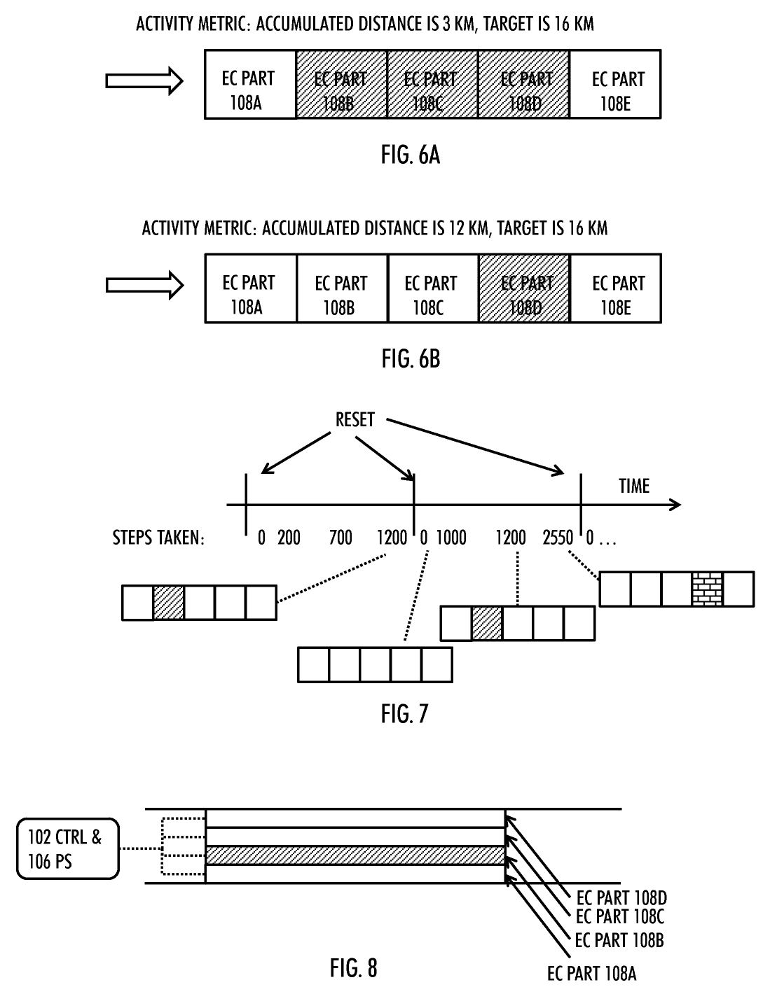

In an embodiment as shown in FIGS. 6A and 6B, the PAMD 100 acquire an indication of a target activity level of the person. This may be set by the user via the UI 110, for example, or there may be a preconfigured target level stored in the memory 104 of the PAMD 100. Then the PAMD 100 may determine whether or not a target activity level is reached. Let us assume, as an example, that the activity metric indicates the accumulated distance. Let us further assume that the set target is 16 km. This 16 km is comprised in the activity zone 306, which is associated with the EC part 108D.

Upon detecting that the target activity level is not reached, the PAMD 100 may control the optical characteristic of each of the electrochromic parts to indicate that the target is not reached. This may be beneficial as then the user easily sees that he/she needs to run/walk/ski more in order to reach the target activity level. Similarly, the accumulated activity metric could indicate an elapsed time of the training, for example, or any other type of activity metric.

There may be a variety of ways on how to show that the target activity level is not yet reached. In one non-limiting example shown in FIGS. 6A and 6B, in the beginning the EC parts 108A-108D have a certain optical state (such as a certain colour). That is, all the EC parts until the EC part 108D corresponding to the target activity level are depicted with a certain optical state. As the person starts running, the acquired activity metric indicates increased accumulated distance. In FIG. 6A, this accumulated distance is 3 kilometres in which case the first EC part 108A may have changed its optical state, whereas the parts 108B-108D may still be in the original optical state. The optical states of the EC parts 108A-108D may change as the activity metric indicates an increased accumulated distance. In FIG. 6B, the accumulated distance is 12 kilometres which is comprised in the activity zone 304 corresponding to the EC part 108C. In this case, the EC parts 108B and 108C may have changed their optical state. However, the EC part 108D may still be e.g. coloured in order to indicate that the target activity level is not yet reached. The EC part 108D may change to being, e.g. transparent only when the target level of 16 km is reached. In another embodiment, there may be predetermined EC part which is highlighted only when the target activity level is not yet reached or only when the target activity level is reached.

In an embodiment, as shown in FIG. 7, the PAMD 100 may reset the accumulated activity level periodically after a predetermined accumulation period. In an embodiment, this predetermined period may be 24 hours, a week, or a month, for example. In FIG. 7 the activity metric indicates an accumulated number of steps taken. The horizontal axis may represent a time domain. As shown, the reset of the accumulated activity level (i.e. reset of the number of steps taken) may take place at the points depicted with vertical lines. This may be beneficial in case a person wants to keep track of the accumulated activity level in a daily basis, for example.

As a result, the person may monitor how many steps he/she has taken on a given day. This may be shown with the EC parts 108A-108E, as explained. For example, the EC part associated with the activity zone comprising the number of accumulated steps taken may be highlighted with respect to the other EC parts, as shown in FIG. 7. As the date changes, the number of accumulated steps taken may be reset and all the EC parts may be, e.g., transparent or black. As the day goes on, the accumulated number of steps taken may increase. At the point where 1200 steps have been taken, the corresponding EC part may be highlighted with respect to the other EC parts. At the end of the current day, the number of steps may be 2550, which may have caused a certain one of the EC parts to change the optical state into a specific colour, for example. Although depicted with respect to number of steps taken, other types of activity metrics indicating accumulated activity levels may be used as well.

In an embodiment, the activity metric represents at least one of the following: heart activity, accumulated time duration of activities above a certain activity threshold, calories consumed, speed, pace, accumulated distance, stride, cadence, power, altitude, accumulated ascent, accumulated descent, incline, decline, temperature, barometric value, recovery level, benefit level, fitness level, training load, accumulated number of jumps, accumulated number of spurts, height of jumps, inactivity level. Each of these activity metric types may be obtained from an appropriate sensor, such as a heart activity sensor, GPS-receiver, foot pod, cadence monitor, accelerometer, or an activity sensor.

For example, let us assume that the activity metric, acquired from an activity sensor, indicates the inactivity level of the person. Let us further assume that this inactivity level indicates that the person has performed, within an inactivity observation period, fewer activities than a predetermined threshold requires. The inactivity level may be indicated with a various different parameters, such as with low elapsed time duration above the activity threshold, low amount of steps taken, low average heart rate, etc. In this case the PAMD 100 may indicate to the user that it is recommended to start performing activities. This indication may take place by the PAMD 100 controlling (e.g. changing) the optical state of a certain EC part. The predetermined threshold may be defined in any physical/physiological value, such as a required number of steps to be taken during the inactivity observation period, such as within a day.

In an embodiment, the PAMD 100 may comprise a default colour selection out of which the user may select the colour to be displayed in a given EC part 108. In an embodiment, a new colour is added to the default set of colours once a given target status level is reached. The target status level may be preconfigured to the PAMD 100 and may be defined with any given type of activity metric, such as amount of calories consumed within one month, for example. There may be a sliding window type of observation window so that as long as the user keeps on performing activities enough, the reached colour level or “status” is maintained (i.e. the accumulated calories may not be reset for this purpose). In another embodiment, the observation window is not of a sliding window type, in which case the accumulated values for this purpose may be reset at the beginning of each observation window, such as n the beginning of each week or month.

In an embodiment, there are EC parts 108 in the PAMD 100 which are not in use or cannot be selected to be in use unless the target status level is first reached. These normally unused EC parts may provide new colours the user may want to use. There may be one or more colours that may be added or taken into use once the target status level is reached. There may be one or more the target status levels, each enabling the usage of a certain colour or colours. These embodiments may provide an additional reason for the user to start performing more activities. For example, the highest target status level may require running 100 km weekly. Once reaching this highest level, a certain EC part may be activated to display a certain colour (such as gold). Once the accumulated distance within the last seven days becomes less than the highest target status, this certain EC part may be changed into transparent state or into a silver state (possibly representing that the second highest target level is still valid), for example.

In an embodiment, the optical state of a certain EC part is changed according to an activity metric indicating a location related activity information. As an example, the EC part may change the optical state at a start point of an activity, at a predefined point of interest, at an end point of an activity. As another example, the optical state of some EC parts may change according to current cardinal direction. For example, the optical state of one EC part may change when the user travels in north.

In an embodiment, the PAMD 100 may further comprise an additional EC part 109, as shown in FIG. 1, or a so-called notification specific EC part and the optical characteristic of that EC part may be controlled on the basis of detecting a need to notify the user. Such notification may be due when an alarm is due, battery level deteriorates, for example.

In an embodiment, the optical state of the additional EC part 109 may change when a measuring reliability, such as the signal level of a GPS signal, deteriorates.

In an embodiment, the optical state of additional EC part 109 may change when a predefined alarm or reminder is due. Such alarm may be due when a battery level drops below a threshold, or a weather forecast (obtainable from Internet, for example) indicates a certain change in the weather, such as an increased UV level. Likewise, the calendar based notes or push notifications may trigger a change in the optical state of an EC part, in case the PAMD 100 has access to calendar notes, for example.

In an embodiment, the optical state of additional EC part 109 may indicate a change of time (e.g. the colour may indicate AM/PM, hour, minute). In an embodiment the optical state of many EC parts may indicate a battery level. In an embodiment, the optical state of an EC part may indicate currently used user interface mode, such as an exercise mode, training history review mode.

In an embodiment, the optical state of additional EC part 109 may indicate whether or not a connection is established between the PAMD 100 and a sensor.

In an embodiment, the user may select a colour or shape to be displayed via any of the EC parts 108, 109 from a user menu via the UI 110.

In an embodiment as shown in FIG. 8, the plurality of EC parts 108A-108D are arranged in a plurality of layers, one on top of another, wherein the EC material of each electrochromic part has a different colour which the EC material can adopt. Each of these EC parts 108A-108D may be able to adopt the optical state of transparency as one optical state option. In an embodiment, when one of the layered EC parts 108A-108D displays a certain colour, then the other layered EC parts 108A-108D (at least the layers on top of the coloured EC part) may be cause to be transparent. Such layered structure may save surface area as one surface area may be used to display several different colours. Further, the display area for the EC part may be very large, as the surface need not necessarily comprise any other EC parts next to this layered structure. Thus, different activity levels indicated by the acquired activity metric may be illustrated at the same surface area of the PAMD 100. The layers may be laminated on top of each other, directly or indirectly (i.e. there may be some transparent material (e.g. glass, plastic) between the layers).

In an embodiment, the EC parts 108A-108D arranged in layers are not of identical shape. For example, the upper layer may be smaller than the lower layer. In such case, different colours may be shown on top of each other so that the background colour is still visible on the edges, for example. In another example, the EC parts 108A-108D arranged in layers are of identical shape. This option may provide ease of implementation.

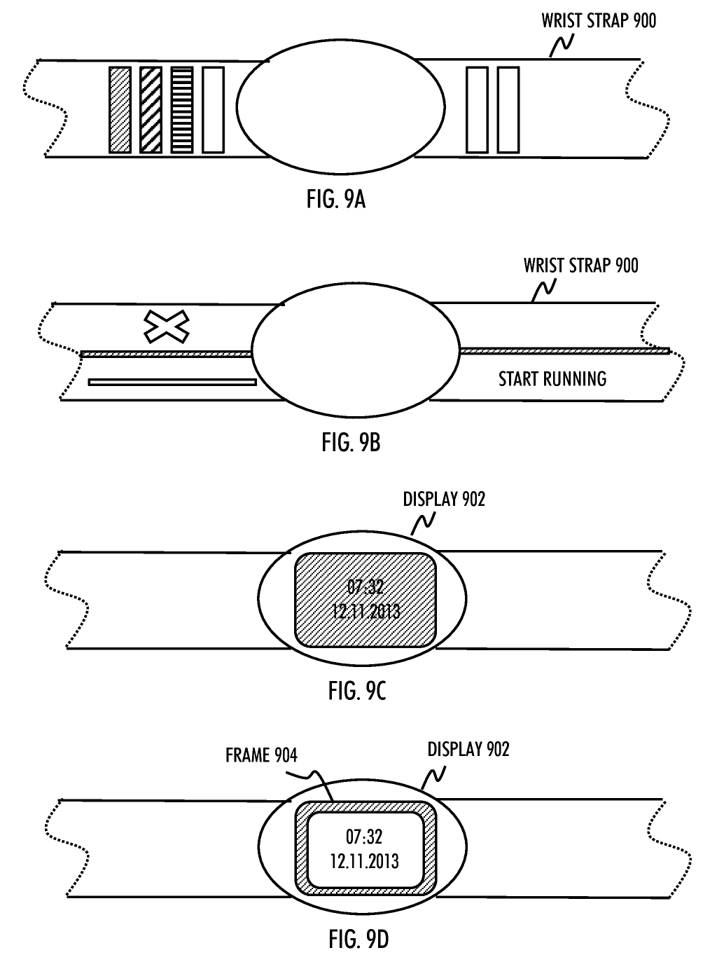

In an embodiment as shown in FIG. 9A, the plurality of EC parts (shown with blocks) is arranged in one layer, one next to another. This embodiment may be combined with the embodiment of FIG. 8 such that many layered structures may be next-to-next. The adjacent EC parts may be directly or indirectly next to each other and may have different or same optical characteristics.

In an embodiment, the at least one EC part is comprised in mechanical structures of the PAMD 100.

In an embodiment, the EC part is integrated into the mechanical structure of the PAMD 100.

In an embodiment, at least one EC part is laminated onto the mechanical structure of the PAMD 100.

In an embodiment, at least one EC part is integrated into the mechanical structure of the PAMD 100 with an adhesive, such as tape or glue.

In an embodiment, at least one EC part is injection molded into the mechanical structure of the PAMD 100.

These mechanical structures may comprise a wrist strap or frames of the display, for example. The wrist strap is significantly larger in surface area than the display. Thus, displaying information on large surface, such as on the wrist strap, may aid the user in observing the information quickly. Especially while a user is exercising it may be beneficial that the user easily and quickly observes the information shown. Looking at the relatively small display and quickly detecting what information is shown may not be easy while running. Thus, showing the information on the mechanical structures, such as on the wrist strap, may be advantageous. The flexibility of the EC materials may allow the implementation of the EC material to structures which may be twisted or bent.

In an embodiment, as shown in FIGS. 9A and 9B, the at least one EC part is comprised in the wrist strap 900 of the PAMD 100. The EC parts may be in one layer or in multiple layers. In an embodiment, the largest dimension of at least one EC part may be parallel to the longitudinal dimension of the wrist strap 900, as is the case in FIG. 9B. These longitudinal EC parts may have a length which is less than the half of the length of the wrist strap 900, a length which is longer than the half of the length of the wrist strap, or a length which is as long as the wrist strap 900. A long EC part may be easy to detect. In an embodiment, the whole wrist strap 900 is made of EC material and serves as a one large EC part.

In an embodiment as shown in FIG. 9C, at least one EC part is comprised in a display 902 of the PAMD 100. The display 902 may be e.g. a LCD display which shows numerical or alphabetical data. However, e.g. the back-ground plate of the display 902 may be of EC material and serve as the EC part 108 so that the background colour of the display 902 may be changed. There may be many EC parts 108 in a layered structure (see FIG. 8) as the background plate so that the background colour may be changed between many different colours, not only between two colours of one EC part. In addition to providing aesthetic benefits, this structure may additionally contribute the easiness of reading the display in different illuminations, for example. In one embodiment, the EC material may be comprised in a lens of the display 902. The EC material used for the lens may be at least partly transparent in each optical state.

In an embodiment as shown in FIG. 9D, the at least one EC part is comprised in at least part of the frame 904 of the display 902 of the PAMD 100. This may be beneficial so that the frame colour may be changed. This may also contribute the easiness of reading the display in different illuminations, in addition to bringing aesthetic benefits, for example.

In an embodiment, the PAMD 100 may comprise a cover on top of the at least one EC parts. The cover may be used to shield the EC parts. The cover may be of transparent material.

In an embodiment, the least one EC part is arranged in a shape of an alphabetic character or a numerical character. For example, as shown in FIG. 9B, the wrist strap 900, or any other part of the PAMD 100, may be caused to display “START TRAINING”, wherein the letters of the command/recommendation are implemented in EC material and serve as the EC part 108 of the PAMD 100. The letters may be transparent when the person is performing enough exercise, whereas the letters may be coloured (by applying the voltage, as explained), when the user is detected to be inactive according to certain criterion. Further, in FIG. 9B it is shown that different shapes of EC parts 108 may be implemented to any of the surfaces of the PAMD 100.

In an embodiment, the shape of at least one EC part is rectangular, or at least substantially rectangular. In an embodiment, the shape of at least one EC part is elliptical. In an embodiment, the shape of at least one EC part is circular.

In an embodiment, the at least one EC part 108 is an integral part of the PAMD 100. In an embodiment, the at least one EC part 108 is an integral part of the mechanical structures of the PAMD 100. In an embodiment, the at least one EC part 108 is an integral part of the casing of the PAMD 100. In an embodiment, the at least one EC part 108 is an integral part of the wrist strap of the PAMD 100. In an embodiment, the at least one EC part 108 is not a part of the display, does not cover the display, and does not affect the appearance of the display of the PAMD 100. In an embodiment, the at least one EC part 108 is outside the display of the PAMD 100. In an embodiment, the at least one EC part 108 is not a part of a liquid crystal display of the PAMD 100.

In an embodiment, a first physical dimension of an EC part 108, looking from the top, is between 1 millimetre and 2 centimetres. In an embodiment, a second physical dimension of the EC part 108, looking from the top, is between 1 millimetre and 15 centimetres.

In an embodiment, the first physical dimension of an EC part 108, looking from the top, is between 2 millimetres and 1 centimetre. In an embodiment, the second physical dimension of the EC part 108, looking from the top, is between 1 centimetre and 5 centimetres.

In an embodiment, the first physical dimension of an EC part 108, looking from the top, is between 2 millimetres and 2 centimetres. In an embodiment, the second physical dimension of the EC part 108, looking from the top, is between 1 millimetre and 1 centimetre.

The physical dimensions of an EC part 108 may be a combination any of the given physical dimension ranges.

The EC material may be flexible. In an embodiment, the EC part 108 may be twisted or bent. The bending may comprise bending the EC part 108 into a roll in either direction. This may allow the implementation of the EC part 108 into a wrist strap or to an organic light emitting diode (OLED) display, for example. The twisting may comprise twisting the EC part in either direction around its longitudinal axis. The EC part 108 may be twisted even 360 degrees around the longitudinal axis within a length of 2 centimetres, for example.

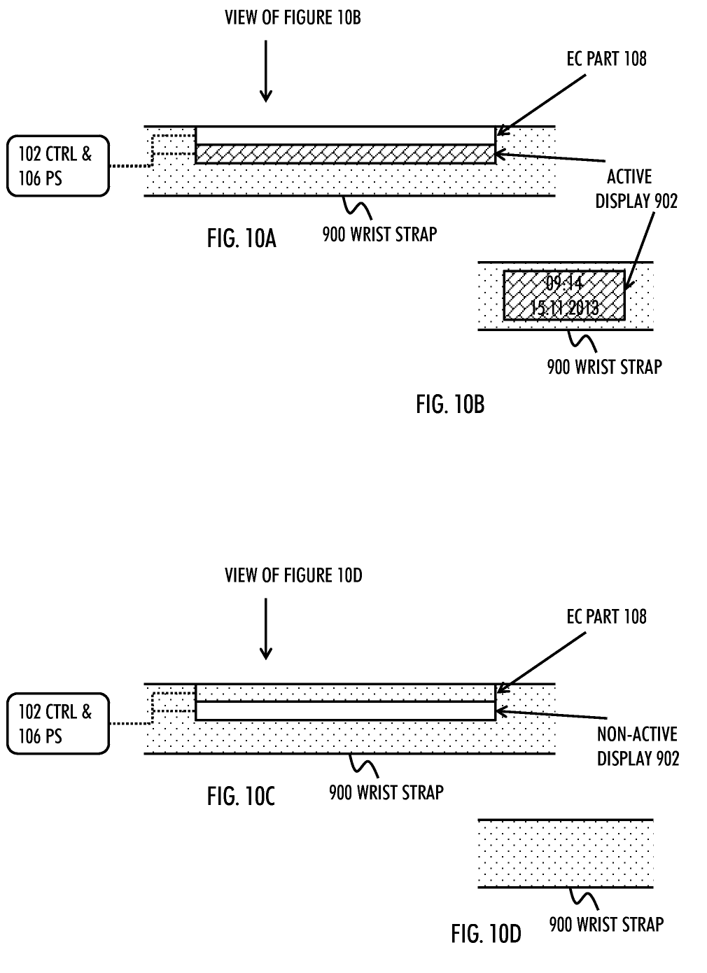

In an embodiment, as shown in FIGS. 10A to 10D, the EC part 108 may cover the display 902 of the PAMD 100. In this example, the control of the optical characteristic of the EC part 108 comprises a reversible change between a first and a second optical state, wherein, in the first state, the EC material of the EC part 108 is transparent and, in the second state, the EC material of the EC part 108 is non-transparent. In the second state, the EC part may adopt substantially the same colour as used in the surroundings of the EC part 108. The first state is shown in FIGS. 10A and 10B, whereas the second state is shown in FIGS. 10C and 10D. FIGS. 10B and 10D show a top-view of FIGS. 10A and 10C, respectively.

In this embodiment, the PAMD 100 may be caused to control the electrochromic part to adopt the first optical state (i.e. transparency) upon activation of the display 902 and to adopt the second optical state (i.e. non-transparency) upon deactivation of the display 902. As shown, the characters on the activated display 902 in FIGS. 10A and 10B are shown to outside when the EC part 108 is controlled to be transparent. The controller 102 may substantially simultaneously change the display into an active mode and change to optical state of the EC part 108 so that the EC part 108 is transparent.

However, when the display is non-active (e.g. shut down), the controller 102 may change to optical state of the EC part 108 so that the EC part 108 is non-transparent. More specifically, the second optical state may comprise that the EC part adopts a colour which is substantially the same colour as is used in the surroundings of the EC part 108. This is shown in FIGS. 100 and 10D in which it is shown that the display 902 becomes invisible to outside and a person looking at the PAMD 100 may not recognize the presence of the display 902. The person looking at the PAMD 100 may only see a flat surface with an even colour.

The embodiment of FIG. 10A to 10D may be beneficial because in case the display 902, such as a LED matrice display, was covered with a thin film of transparent non-EC material, the display 902 could at least faintly reflect through the transparent non-EC material (such as plastic) even when the display is non-active. This may not be desirable from the aesthetic point of view. If the plastic cover is made thicker, so that the display 902 becomes totally invisible when non-active, the thick cover may cause diffraction problems when the display is turned into an active mode. Thus, providing the covering of the display 902 with the proposed EC material (EC part 108) may be advantageous, because it may allow using a thin layer of the EC part on top of the display 902, which may be good from the aesthetic point of view as well as for effectively covering the display 902 from external factors.

In an embodiment, to further improve the illustration of what is shown on the display 902, the EC part 108 covering the display 108 may comprise apertures, wherein the apertures are arranged to overlap with the LEDs of the LED matrice display 902. When the display 902 is non-active and the EC part 108 adopts the non-transparent, second state (as in FIGS. 10C and 10D), the small holes in the EC part 108 may still naturally be open. However, as the holes are small (e.g. each corresponding to the size of a pixel of the display), the appearance of the PAMD 100 may still be improved compared to the case where the display 902 is covered with a non-EC material.

Regardless of whether the plurality of EC parts is arranged in a single layer or in a plurality of layers, the EC parts may be formed on or in the PAMD 100 according to any of the embodiments described.

Looking further of the functional entities of FIG. 1, the memory 104 may be implemented using any suitable data storage technology, such as semiconductor based memory devices, flash memory, magnetic memory devices and systems, optical memory devices and systems, fixed memory and removable memory. The TRX 112 may comprise hardware and/or software for realizing communication connectivity according to one or more communication protocols. The communication connectivity may be to at least one physical/physiological sensor, to Internet, to a computer, for example.

The control circuitry 102 may comprise an activity metric analysis circuitry 102A for analysing the acquired activity metric. An EC part control circuitry 102B may be for controlling the optical characteristics of the at least one EC part, according to any of the embodiments.

As used in this application, the term ‘circuitry’ refers to all of the following: (a) hardware-only circuit implementations, such as implementations in only analog and/or digital circuitry, and (b) combinations of circuits and software (and/or firmware), such as (as applicable): (i) a combination of processor(s) or (ii) portions of processor(s)/software including digital signal processor(s), software, and memory(ies) that work together to cause an apparatus to perform various functions, and (c) circuits, such as a microprocessor(s) or a portion of a microprocessor(s), that require software or firmware for operation, even if the software or firmware is not physically present. This definition of ‘circuitry’ applies to all uses of this term in this application. As a further example, as used in this application, the term ‘circuitry’ would also cover an implementation of merely a processor (or multiple processors) or a portion of a processor and its (or their) accompanying software and/or firmware. The term ‘circuitry’ would also cover, for example and if applicable to the particular element, a baseband integrated circuit or applications processor integrated circuit for a mobile phone or a similar integrated circuit in a server, a cellular network device, or another network device.

The techniques and methods described herein may be implemented by various means. For example, these techniques may be implemented in hardware (one or more devices), firmware (one or more devices), software (one or more modules), or combinations thereof. For a hardware implementation, the apparatus(es) of embodiments may be implemented within one or more application-specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. For firmware or software, the implementation can be carried out through modules of at least one chip set (e.g. procedures, functions, and so on) that perform the functions described herein. The software codes may be stored in a memory unit and executed by processors. The memory unit may be implemented within the processor or externally to the processor. In the latter case, it can be communicatively coupled to the processor via various means, as is known in the art. Additionally, the components of the systems described herein may be rearranged and/or complemented by additional components in order to facilitate the achievements of the various aspects, etc., described with regard thereto, and they are not limited to the precise configurations set forth in the given figures, as will be appreciated by one skilled in the art.

Embodiments as described may also be carried out in the form of a computer process defined by a computer program. The computer program may be in source code form, object code form, or in some intermediate form, and it may be stored in some sort of carrier, which may be any entity or device capable of carrying the program. For example, the computer program may be stored on a computer program distribution medium readable by a computer or a processor. The computer program medium may be, for example but not limited to, a record medium, computer memory, read-only memory, electrical carrier signal, telecommunications signal, and software distribution package, for example. Coding of software for carrying out the embodiments as shown and described is well within the scope of a person of ordinary skill in the art.

Even though the invention has been described above with reference to an example according to the accompanying drawings, it is clear that the invention is not restricted thereto but can be modified in several ways within the scope of the appended claims. Therefore, all words and expressions should be interpreted broadly and they are intended to illustrate, not to restrict, the embodiment. It will be obvious to a person skilled in the art that, as technology advances, the inventive concept can be implemented in various ways. Further, it is clear to a person skilled in the art that the described embodiments may, but are not required to, be combined with other embodiments in various ways.

What is claimed is:

1. A portable physical activity monitor device, comprising:
a power source for providing power to the physical activity monitor device;
at least one processor and at least one memory including a computer program code, wherein the at least one memory and the computer program code are configured, with the at least one processor, to cause the physical activity monitor device to perform operations comprising:
acquiring an indication of a physical activity metric of a person carrying the physical activity monitor device,
wherein the physical activity monitor device further comprises at least one electrochromic part comprising bi-stable electrochromic material, and the physical activity monitor device is further caused to perform operations comprising:
controlling an optical characteristic of the electrochromic part on the basis of the acquired activity metric.

2. The physical activity monitor device of claim 1, wherein the physical activity monitor device comprises a plurality of electrochromic parts, each comprising bi-stable electrochromic material, and the physical activity monitor device is further caused to perform operations comprising:
defining a plurality of activity zones, wherein each activity zone comprises a different range of activity metric values;
associating each activity zone with a different electrochromic part;
detecting to which of the plurality of predetermined activity zones the acquired activity metric belongs to;
identifying the electrochromic part which is associated with the activity zone comprising the acquired activity metric; and
controlling the optical characteristic of each of the plurality of electrochromic parts on the basis of which of the plurality of electrochromic parts is the identified electrochromic part.

3. The physical activity monitor device of claim 2, wherein the physical activity monitor device is further caused to perform operations comprising:
identifying the type of the activity metric;
redefining the plurality of activity zones on the basis of the identified type of activity metric; and
redefining the associations between the electrochromic parts and the activity zones.

4. The physical activity monitor device of claim 2, wherein the activity metric represents an accumulated activity level and each of the plurality of activity zones comprises a different range of accumulated activity levels, and the physical activity monitor device is further caused to perform operations comprising:
controlling the optical characteristic of each of the plurality of electrochromic parts on the basis of what the accumulated activity level is.

5. The physical activity monitor device of claim 4, wherein the physical activity monitor device is further caused to perform operations comprising:
controlling the optical characteristics of the electrochromic parts such that the electrochromic part, which corresponds to the activity zone within which the current accumulated activity level is, adopts a different optical state than the rest of the electrochromic parts.

6. The physical activity monitor device of claim 4, wherein the physical activity monitor device is further caused to perform operations comprising:
controlling the optical characteristics of the electrochromic parts such that the at least one electrochromic part, which corresponds to the lower or higher accumulated activity levels than the current accumulated activity level, adopts a different optical state than the rest of the electrochromic parts.

7. The physical activity monitor device of claim 4, wherein the physical activity monitor device is further caused to perform operations comprising:
resetting the accumulated activity level periodically after a predetermined accumulation period.

8. The physical activity monitor device of claim 1, wherein the physical activity monitor device is further caused to perform operations comprising:
acquiring an indication of a target activity level of the person;
determining whether or not a target activity level is reached; and
upon detecting that the target activity level is not reached, controlling the optical characteristic of each of the electrochromic parts to indicate that the target is not reached.

9. The physical activity monitor device of claim 1, wherein the activity metric represents at least one of the following: heart activity, accumulated time duration of activities above a certain activity threshold, calories consumed, speed, pace, accumulated distance, stride, cadence, power, altitude, accumulated ascent, accumulated descent, incline, decline, temperature, barometric value, recovery level, benefit level, fitness level, training load, accumulated number of jumps, accumulated number of spurts, height of jumps, inactivity level.

10. The physical activity monitor device of claim 1, wherein the physical activity monitor device comprises a plurality of electrochromic parts arranged in a plurality of layers, one on top of another, wherein the electrochromic material of each electrochromic part has a different colour which the electrochromic material can adopt.

11. The physical activity monitor device of claim 1, wherein the physical activity monitor device comprises a plurality of electrochromic parts arranged in one layer, one next to another.

12. The physical activity monitor device of claim 1, wherein the electrochromic part covers a display of the physical activity monitor device, and the control of the optical characteristic comprises a reversible change between a first and a second optical state, wherein, in the first state, the electrochromic material is transparent and, in the second state, the electrochromic material is non-transparent and the physical activity monitor device is further caused to perform operations comprising:
controlling the electrochromic part to adopt the first optical state upon activation of the display and to adopt the second optical state upon deactivation of the display.

13. The physical activity monitor device of claim 1, wherein at least one electrochromic part is comprised in a mechanical structure of the physical activity monitor device.

14. The physical activity monitor device of claim 1, wherein at least one electrochromic part is comprised in a wrist strap of the physical activity monitor device.

15. The physical activity monitor device of claim 1, wherein at least one electrochromic part is comprised in at least part of the frame of a display of the physical activity monitor device.

16. The physical activity monitor device of claim 1, wherein at least one electrochromic part is arranged in a shape of an alphabetic character or a numerical character.

17. A computer program product embodied on a non-transient distribution medium readable by a computer and comprising program instructions which, when executed by a portable physical activity monitor device, cause the physical activity monitor device to perform operations comprising:
acquiring an indication of a physical activity metric of a person carrying the physical activity monitor device, wherein the physical activity monitor device comprises at least one electrochromic part comprising bi-stable electrochromic material; and
controlling an optical characteristic of the electrochromic part on the basis of the acquired activity metric.
